(19) United States
(12) Patent Application Publication
(10) Pub. No.: US 2024/0285589 A1
(43) Pub. Date: Aug. 29, 2024

(54) TREATMENT USING AN ANTIVIRAL COMPOUND AND NITAZOXANIDE

(71) Applicant: Theodore HENDERSON, Centennial, CO (US)

(72) Inventor: Theodore HENDERSON, Centennial, CO (US)

(21) Appl. No.: 18/564,955

(22) PCT Filed: May 28, 2021
(86) PCT No.: PCT/US2021/034818

§ 371 Date: Nov. 28, 2023

(51) Int. Cl. A61K 31/426; A61K 31/52; A61K 31/522; A61P 31/12

(52) U.S. Cl. CPC A61K 31/426; A61K 31/52; A61K 31/522; A61P 31/12

(57) ABSTRACT

The disclosure relates to methods for treating a virally induced functional disorder (VIFD), a functional somatic syndrome (FSS) and/or a herpes-family virus by administering a therapeutically-effective combination of an antiviral compound and nitazoxanide. The disclosure is further related to pharmaceutical compositions comprising a combination of an antiviral compound and nitazoxanide.

### FIELD OF THE DISCLOSURE

The disclosure relates to methods for treating a virally induced functional disorder (VIFD), a functional somatic syndrome (FSS) and/or a herpes-family virus by administering a therapeutically-effective combination of an antiviral compound and nitazoxanide. The disclosure is further related to pharmaceutical compositions comprising a combination of an antiviral compound and nitazoxanide.

### BACKGROUND

Virally induced functional disorders (VIFD) are disorders with a viral etiology. There are many disorders that viruses have been evidenced to play a role. In particular, the Herpes family of DNA viruses has been found to be associated or have a causal role in many disorders. For example, multiple studies have shown that chronic fatigue syndrome (CFS) patients have a high incidence of Herpes family virus latent or chronic infections. Similarly, extensive laboratory and clinical research has shown Herpes 1, the cold sore virus, has a key role in the development of Alzheimer's disease. Thus, the literature teaches that VIFD may share common causal factors.

Functional disorders involve one or more organ systems, have obscure or unsettled etiologies, and impair the person's function. Subsumed under a category of functional somatic syndromes (FSS), these multiple disorders share an overlap of bodily complaints, functional disturbance in a bodily system, and fatigue. FSS may be defined as conditions “characterized by patterns of persistent bodily complaints for which adequate examination does not reveal sufficiently explanatory structural or other specified pathology” [P. Henningsen, et al. (2007) Lancet 369, 946-954]. A large proportion of persons afflicted with one FSS have additional symptoms or full criteria for additional FSS disorders. For example, 20-65% of persons with irritable bowel syndrome (IBS) have fibromyalgia, 36-63% have CFS, and 16% have temporomandibular joint disorder (Wiehle and Henningsen, 2014). Similarly, 35-92% of CFS patients are afflicted with IBS (Wiehle and Henningsen, 2014).

There remains an unmet need for effective therapeutic options for virally induced functional disorders and functional disorders.

### SUMMARY

Provided herein is a method of treating a virally induced functional disorder (VIFD) in a subject in need thereof, the method comprising administering to the subject a therapeutically effective amount of each of an antiviral compound and nitazoxanide.

Further provided herein is a method of treating a functional somatic syndrome (FSS) in a subject in need thereof, the method comprising administering to the subject a therapeutically effective amount of each of an antiviral compound and nitazoxanide.

Also provided herein is a method of treating a Herpes-family virus in a subject in need thereof, the method comprising administering to the subject a therapeutically effective amount of each of an antiviral compound and nitazoxanide.

Also provided herein is a pharmaceutical composition comprising a therapeutically effective amount of each of an antiviral compound and nitazoxanide.

### BRIEF DESCRIPTION OF THE DRAWINGS

FIGS. 1A-1B show perfusion SPECT scan from Case Study 3. FIG. 1A shows diffuse hypoperfusion consistent with infectious or toxic injury to the brain in the patient prior to treatment. FIG. 1B shows that a repeat perfusion SPECT brain scan was performed after 8 months of combination therapy that revealed marked improvement in brain function and near-complete resolution of the pathological findings associated with infectious injury. Scale illustrates levels of perfusion with normal perfusion at the central light grey and decreased perfusion illustrated by progressively darker shades of grey. The figure further shows the patient's handwriting before and after treatment with combination therapy.

FIG. 2 is a diagram of virally-induced functional disorders.

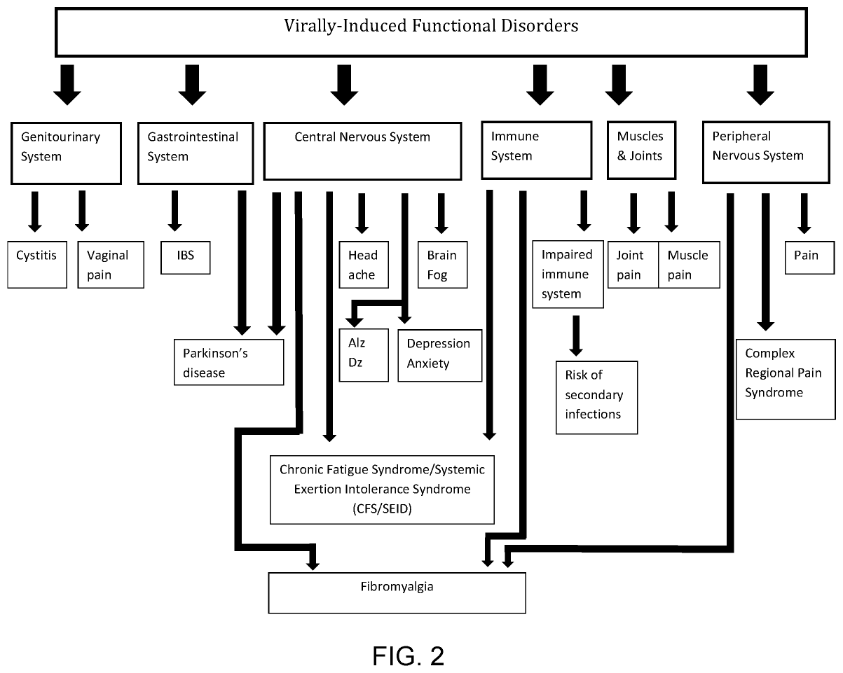

FIGS. 3A-3B shows a SPECT scan (A) before treatment and (B) after treatment for the patient disclosed in Example 6. The SPECT scans show a left lateral view and a superior (looking down) view. FIG. 3A (before) shows extensive areas of decreased function (shown in white). FIG. 3B (after) shows marked improvement in brain function based on a statistical comparison to a normative database. Scale illustrates levels of perfusion in a different manner from FIG. 1. The dark grey central portion of the scale illustrates statistically normal perfusion in a given area relative to a normative database. Areas of progressively lighter grey to white illustrate areas which are 2 standard deviation (SD), 3 SD, and 4 SD below the mean of the normative database, respectively.

### DETAILED DESCRIPTION

The disclosure relates to methods for treating a virally induced functional disorder (VIFD), a functional somatic syndrome (FSS) and/or a herpes-family virus by administering a therapeutically-effective combination of an antiviral compound and nitazoxanide. The disclosure is further related to pharmaceutical compositions comprising a combination of an antiviral compound and nitazoxanide.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

In some embodiments provided herein is a method for treating a virally induced functional disorder (VIFD) in a subject in need thereof, the method comprising administering to the subject a therapeutically effective amount of each of an antiviral compound and nitazoxanide.

In some embodiments provided herein is a method for treating a functional somatic syndrome (FSS) in a subject in need thereof, the method comprising administering to the subject a therapeutically effective amount of each of an antiviral compound and nitazoxanide.

In some embodiments provided herein is a method for treating a Herpes-family virus in a subject in need thereof, the method comprising administering to the subject a therapeutically effective amount of each of an antiviral compound and nitazoxanide.

In some embodiments, the antiviral compound is an antiviral guanine analog. Antiviral agents used to treat for example herpes family viruses are typically antiviral guanine analogs. These agents inhibit viral replication by substituting for guanine at the viral thymidine kinase enzyme. This leads to failure of DNA replication through their competitive inhibition of the viral DNA polymerase and termination of DNA replication. All Herpes family viruses share similar viral DNA polymerases and hence the antiviral guanine analogs are effective against all members of the Herpes family: Herpes simplex 1 (Herpes 1), genital herpes (Herpes 2), varicella-zoster (Herpes 3), Epstein-Barr virus (Herpes 4), cytomegalovirus (Herpes 5), Human Herpes 6 (Herpes 6), Human Herpes 7 (Herpes 7), and Human Herpes 8 (Herpes 8), as well as any unnamed or unidentified Herpes viruses. In some embodiments, the antiviral compound is valacyclovir, acyclovir, famciclovir, gangciclovir, or valganciclovir.

Nitazoxanide (NTZ), or 2-(acetyloxy)-N-(5-nitro-2-thazolyl) benzamide, is a thiazolide broad-spectrum anti-infective. NTZ's active metabolite, tizoxamide, is also effective against a variety of infectious agents.

In particular embodiments, the virally induced functional disorder (VIFD) is a disorder of the genitourinary system, including, but not limited to cystitis and vaginal pain. In particular embodiments, the VIFD is a disorder of the gastrointestinal system, including but not limited to IBS, ulcerative colitis, and colitis. In particular embodiments, the VIFD is a disorder of the central nervous system, including, but not limited to, headache, brain fog, Parkinson's disease, Alzheimer's disease, dementia, depression, anxiety, and CFS. In particular embodiments, the VIFD is a disorder of the immune system, including but not limited to immune system impairment, CFS, and fibromyalgia. In particular embodiments, the VIFD is a disorder of the musculoskeletal system, including but not limited to joint pain, muscle pain, and weakness. In particular embodiments, the VIFD is a disorder of the peripheral nervous system including pain, numbness, and complex regional pain syndrome.

Functional somatic syndrome (FSS) as used herein is defined as a condition “characterized by patterns of persistent bodily complaints for which adequate examination does not reveal sufficiently explanatory structural or other specified pathology.” The methods disclosed herein can be used to treat FSS including but not limited to: IBS, CFS, fibromyalgia, multiple chemical sensitivity, muscle pain, joint pain, nonspecific chest pain, premenstrual syndrome, non-ulcer dyspepsia, repetitive strain injury, headache, temporomandibular joint disorder, chronic pain, chronic pelvic pain, atypical facial pain, low back pain, sick building syndrome, Gulf War syndrome, interstitial cystitis, chronic Lyme disease, depression, post-traumatic stress disorder (PTSD), chronic anxiety disorder, brain fog or cognitive dysfunction, unrefreshing sleep, insomnia, dizziness and tinnitus.

The term “subject” is intended to include human and non-human animals, particularly mammals.

The terms “treatment” or “treat” as used herein refer to both therapeutic treatment and prophylactic or preventative measures.

The terms “administration” or “administering” as used herein refer to providing, contacting, and/or delivering a compound or compositions by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, or using implants.

The terms “co-administered” or “in combination” as used herein refer to simultaneous or sequential administration of multiple compounds or compositions. A first compound or composition may be administered before, concurrently with, or after administration of a second compound or composition. The first compound or composition and the second compound or composition may be simultaneously or sequentially administered on the same day, or may be sequentially administered within 1 day, 2 days, 3 days, 4 days, 5 days, 6 days, 1 week, 2 weeks, 3 weeks, or 1 month of each other. In some embodiments, compounds or compositions are co-administered during the period in which each of the compounds or composition are exerting at least some physiological effect and/or has remaining efficacy.

A “therapeutically effective dose” or “therapeutic dose” is an amount sufficient to effect desired clinical results (i.e., achieve therapeutic efficacy). A therapeutically effective dose can be administered in one or more administrations.

The dose of the antiviral compound will vary depending, in part, upon the size (body weight, body surface, or organ size) and condition (the age and general health) of the subject. In one embodiment, the amount of antiviral compound is present in a unit dosage form from about 250 mg to about 6000 mg. In one embodiment, the amount of antiviral compound is present in a unit dosage form from about 250 mg to about 4000 mg. In one embodiment, the amount of antiviral compound is present in a unit dosage form from about 250 mg to about 2000 mg. In one embodiment, the amount of antiviral compound is present in a unit dosage form of 1000 mg. In one embodiment, the amount of antiviral compound is present in a unit dosage form of 2000 mg.

In one embodiment, the amount of famciclovir is present in a unit dosage form from about 250 mg to about 4000 mg. In another embodiment, the amount of famciclovir is present in a unit dosage form of about 250 mg or about 500 mg. In another embodiment, the amount of famciclovir is present in a unit dosage form from about 125 mg to about 500 mg. In another embodiment, the amount of famciclovir is present in a unit dosage form from about 500 mg to about 1000 mg. In another embodiment, the amount of famciclovir is present in a unit dosage form about 125 mg, about 250 mg, about 500 mg or about 1000 mg. In another embodiment, the amount of famciclovir is present in a unit dosage form of 500 mg. In another embodiment, the amount of famciclovir is present in a unit dosage form of 1000 mg.

In another embodiment, the amount of valacyclovir is present in a unit dosage form from about 1000 mg to about 6000 mg. In another embodiment, the amount of valacyclovir is present in a unit dosage form of 1000 mg. In another embodiment, the amount of valacyclovir is present in a unit dosage form of 2000 mg. In another embodiment, the amount of valganciclovir is present in a unit dosage form from about 400 mg to about 1800 mg. In another embodiment, the amount of valganciclovir is present in a unit dosage form of 450 mg. In another embodiment, the amount of valganciclovir is present in a unit dosage form of 900 mg.

The dose of nitazoxanide will vary depending, in part, upon the size (body weight, body surface, or organ size) and condition (the age and general health) of the subject. In one embodiment, the amount of NTZ is present in a unit dosage form from about 10 mg to about 2000 mg. In one embodiment, the amount of NTZ is present in a unit dosage form from about 50 mg to about 1000 mg. In one embodiment, the amount of NTZ is present in a unit dosage form from about 10 mg to about 500 mg. In one embodiment, the amount of NTZ is present in a unit dosage form from about 100 mg to about 500 mg. In one embodiment, the amount of NTZ is present in a unit dosage form from about 500 mg to about 1000 mg. In some embodiments, the amount of NTZ is present in a unit dosage form of about 100 mg, about 500 mg, about 700 mg or about 1000 mg. In some embodiments, the amount of NTZ is present in a unit dosage form of 500 mg.

In one embodiment, the antiviral compound and NTZ is administered in a dose weight ratio range from about one-to-one to about five hundred-to-one of the antiviral compound to NTZ. In one embodiment, the antiviral compound and NTZ is administered in a dose weight ratio range from about one-to-one to about one hundred-to-one of the antiviral compound to NTZ. In one embodiment, the antiviral compound and NTZ is administered in a dose weight ratio range from about one-to-one to about fifty-to-one of the antiviral compound to NTZ. In one embodiment, the antiviral compound and NTZ is administered in a dose weight ratio range from about one-to-one to about twenty-to-one of the antiviral compound to NTZ. In one embodiment, the antiviral compound and NTZ is administered in a dose weight ratio range from about one-to-one to about five-to-one of the antiviral compound to NTZ.

In one embodiment, the antiviral compound and NTZ are co-administered twice daily to a subject, wherein the amount of the antiviral compound is present in a unit dosage form of 450 mg, and wherein the amount of NTZ is present in a unit dosage form of 500 mg. In one embodiment, the antiviral compound and NTZ are co-administered twice daily to a subject, wherein the amount of the antiviral compound is present in a unit dosage form of 500 mg, and wherein the amount of NTZ is present in a unit dosage form of 500 mg. In one embodiment, the antiviral compound and NTZ are co-administered twice daily to a subject, wherein the amount of the antiviral compound is present in a unit dosage form of 900 mg, and wherein the amount of NTZ is present in a unit dosage form of 500 mg. In one embodiment, the antiviral compound and NTZ are co-administered twice daily to a subject, wherein the amount of the antiviral compound is present in a unit dosage form of 1000 mg, and wherein the amount of NTZ is present in a unit dosage form of 500 mg. In one embodiment, the antiviral compound and NTZ are co-administered twice daily to a subject, wherein the amount of the antiviral compound is present in a unit dosage form of 2000 mg, and wherein the amount of NTZ is present in a unit dosage form of 500 mg. In one embodiment, the antiviral compound and NTZ are co-administered twice daily to a subject, wherein the first administration of the antiviral compound is a unit dosage form of 1000 mg and the second administration of the antiviral compound is a unit dosage form of 2000 mg, and wherein the amount of NTZ is present in a unit dosage form of 500 mg. In one embodiment, the antiviral compound is valacyclovir. In one embodiment, the antiviral compound is famciclovir. In one embodiment, the antiviral compound is valacyclovir. In one embodiment, the antiviral compound is valganciclovir.

In one embodiment, famciclovir and NTZ are co-administered to a subject, wherein the amount of famciclovir is present in a unit dosage form from about 250 mg to about 1000 mg, and wherein the amount of NTZ is present in a unit dosage form from about 100 mg to about 1000 mg.

In some embodiments, the antiviral compound is administered once a day. In another embodiment, the antiviral compound is administered twice a day. In another embodiment, the antiviral compound is administered more than twice a day. In some embodiments, when administered more than once a day, the unit dosage form of the antiviral compound is the same at each administration. In another embodiment, when administered more than once a day, the unit dosage form of the antiviral compound is different at each administration. In some embodiments, the unit dosage form of the antiviral compound is 500 mg. In some embodiments, the unit dosage form of the antiviral compound is 1000 mg. In some embodiments, the unit dosage form of the antiviral compound is 2000 mg.

In some embodiments, NTZ is administered once a day. In another embodiment, NTZ is administered twice a day. In another embodiment, NTZ is administered more than twice a day. In some embodiments, when administered more than once a day, the unit dosage form of NTZ is the same at each administration. In another embodiment, when administered more than once a day, the unit dosage form of NTZ is different at each administration. In some embodiments, the unit dosage form of NTZ is 500 mg. In some embodiments, the unit dosage form of NTZ is 250 mg. In some embodiments, the unit dosage form of NTZ is 1000 mg.

In some embodiments, the antiviral compound and NTZ are administered over a two week treatment period, over a four week treatment period, over a six week treatment period, over an eight week treatment period, over a twelve-week treatment period, over a twenty-four week treatment period, over a thirty-six week treatment period, over a forty-eight week treatment period, over a sixty week treatment period over a seventy-two week treatment period, or over a one year or more treatment period. In another embodiment the antiviral compound and NTZ is administered is administered chronically or continuously over long periods of time (months to years).

In particular embodiments the methods, as described herein, result in a reduction in the administration of at least one additional therapeutic compound previously administered to the subject. In some embodiments, the compound previously administered to the subject is gabapentin, clonazepam, pregabalin, duloxetine, milnacipran, amitriptyline, fluoxetine, paroxetine, sertraline, citalopram, escitalopram, levodopa, carbidopa, pramipexole, ropinirole, donepezil, galantamine, menantine, COX-2 inhibitor, non-steroidal anti-inflammatories, tramadol, morphine, sleep aids and/or muscle relaxers.

The terms “pharmaceutical composition” or “therapeutic composition” as used herein refer to a compound or composition capable of inducing a desired therapeutic effect when properly administered to a subject.

The terms “pharmaceutically acceptable carrier” or “physiologically acceptable carrier” as used herein refer to one or more formulation materials suitable for accomplishing or enhancing the delivery of one or more antibodies of the disclosure.

In one embodiment provided herein is a pharmaceutical composition, comprising a pharmaceutically acceptable carrier in combination with a therapeutically effective amount of an antiviral compound and a therapeutically-effective amount of NTZ.

In some embodiments, the antiviral compound in the pharmaceutical composition is an antiviral guanine analog. In some embodiments, the antiviral compound is valacyclovir, acyclovir, famciclovir, gangciclovir, or valganciclovir.

In particular embodiments, the weight ratio range of the antiviral compound to NTZ in the pharmaceutical composition is from about one-to-one to about five hundred-to-one. In particular embodiments, the weight ratio range of the antiviral compound to NTZ is about one hundred-to-one, about fifty-to-one, about twenty-to-one, or about five-to-one.

In one embodiment, the amount of antiviral compound is present in the pharmaceutical composition in a unit dosage form from about 250 mg to about 6000 mg. In one embodiment, the amount of antiviral compound is present in a unit dosage form from about 250 mg to about 4000 mg. In one embodiment, the amount of antiviral compound is present in a unit dosage form from about 250 mg to about 2000 mg. In one embodiment, the amount of antiviral compound is present in a unit dosage form of 1000 mg. In one embodiment, the amount of antiviral compound is present in a unit dosage form of 2000 mg.

In one embodiment, the amount of famciclovir is present in the pharmaceutical composition in in a unit dosage form from about 250 mg to about 4000 mg. In another embodiment, the amount of famciclovir is present in a unit dosage form of about 250 mg or about 500 mg. In another embodiment, the amount of famciclovir is present in a unit dosage form from about 125 mg to about 500 mg. In another embodiment, the amount of famciclovir is present in a unit dosage form from about 500 mg to about 1000 mg. In another embodiment, the amount of famciclovir is present in a unit dosage form about 125 mg, about 250 mg, about 500 mg or about 1000 mg. In another embodiment, the amount of famciclovir is present in a unit dosage form of 500 mg. In another embodiment, the amount of famciclovir is present in a unit dosage form of 1000 mg.

In another embodiment, the amount of valacyclovir is present in the pharmaceutical composition in a unit dosage form from about 1000 mg to about 6000 mg. In another embodiment, the amount of valacyclovir is present in a unit dosage form from about 1000 mg to about 6000 mg. In another embodiment, the amount of valacyclovir is present in a unit dosage form of 1000 mg. In another embodiment, the amount of valacyclovir is present in a unit dosage form of 2000 mg. In another embodiment, the amount of valganciclovir is present in a unit dosage form from about 400 mg to about 1800 mg. In another embodiment, the amount of valganciclovir is present in a unit dosage form of 450 mg. In another embodiment, the amount of valganciclovir is present in a unit dosage form of 900 mg.

In one embodiment, the amount of NTZ is present in the pharmaceutical composition in a unit dosage form from about 10 mg to about 2000 mg. In one embodiment, the amount of NTZ is present in a unit dosage form from about 50 mg to about 1000 mg. In one embodiment, the amount of NTZ is present in a unit dosage form from about 10 mg to about 500 mg. In one embodiment, the amount of NTZ is present in a unit dosage form from about 100 mg to about 500 mg. In one embodiment, the amount of NTZ is present in a unit dosage form from about 500 mg to about 1000 mg. In some embodiments, the amount of NTZ is present in a unit dosage form of about 100 mg, about 500 mg, about 700 mg or about 1000 mg. In some embodiments, the amount of NTZ is present in a unit dosage form of 500 mg. In some embodiments, the unit dosage form of NTZ is 250 mg. In some embodiments, the unit dosage form of NTZ is 1000 mg.

The compositions of the disclosure may be administered orally, including by swallowing, so that the composition enters the gastrointestinal tract, or absorbed into the blood stream directly from the mouth (e.g., buccal or sublingual administration). Suitable compositions for oral administration include solid formulations such as tablets, lozenges and capsules, which can contain liquids, gels, or powders.

Compositions for oral administration may be formulated as immediate or modified release, including delayed or sustained release, optionally with enteric coating.

Liquid formulations can include solutions, syrups and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Tablets may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch and the like.

Suitable lubricants, for use in a tablet, may be present in amounts from about 0.1% to about 5% by weight, and include calcium, zinc or magnesium stearate, sodium stearyl fumarate and the like.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol and starch.

Suitable surface active agents and glidants, for use in a tablet, may be present in amounts from about 0.1% to about 3% by weight, and include polysorbate 80, sodium dodecyl sulfate, talc and silicon dioxide.

Compositions of the disclosure may be administered directly into the blood stream, muscle, or internal organs. Suitable means for parenteral administration include intravenous, intra-muscular, subcutaneous intra-arterial, intraperitoneal, intrathecal, intracranial, and the like. Suitable devices for parenteral administration include injectors (including needle and needle-free injectors) and infusion methods. Compositions for parenteral administration may be formulated as immediate or modified release, including delayed or sustained release. Most parenteral formulations are aqueous solutions containing excipients, including salts, buffering agents and carbohydrates. Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

Compositions of the disclosure may be administered topically to the skin or transdermally. Formulations for this topical administration can include lotions, solutions, creams, gels, hydrogels, ointments, foams, implants, patches and the like. Pharmaceutically acceptable carriers for topical administration formulations can include water, alcohol, mineral oil, glycerin, polyethylene glycol and the like. Topical administration can also be performed by electroporation, iontophoresis, phonophoresis and the like. Compositions for topical administration may be formulated as immediate or modified release, including delayed or sustained release.

In some embodiments provided herein is a kit comprising an antiviral compound and NTZ in association with each other, as in a single package or in a drug dispensing device. Such kits, used by a subject, may be dispensed by a hospital-formulary, retail pharmacist, or prescribing-physician.

In one embodiment, the kit comprises a single package, with therapeutically-effective doses of an antiviral compound and NTZ, in the form of tablets or capsules in separate containers (e.g., bottles) held separately, as in a tray, and bound together in a single package using, for example, shrink wrap, tape, or a plastic or cardboard box enclosing the components.

In another embodiment, separate, therapeutically-effective doses of an antiviral compound and NTZ, in the form of tablets or capsules, are co-packaged, in a single blister pack.

In another embodiment, the kit comprises an antiviral compound and NTZ, in the form of tablets or capsules, which are co-dispensed from a device which delivers the components from a storage receptacle using, for example, one or more levers to co-dispense individual dose forms of an antiviral compound and NTZ to be administered in combination.

The kit is also used for parenteral administration of dose forms of an antiviral compound and NTZ. For example, individual doses of an antiviral compound and NTZ in the form of lyophilized powders, either separately or with an antiviral compound and NTZ mixed together in therapeutically-effective doses, arranged in a package also comprising separately contained vials of sterile water or buffer solution, and optionally a sterile packaged syringe for administration of the dose combination following dissolution.

The kit may further comprise directions, in compliance with approved instructions from a government agency (e.g., U.S. FDA), on how to use the kit components suitable for administration to obtain a therapeutic outcome.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only, and should not be construed as limiting the scope of the invention in any way.

### Example 1: Case Study 1

An adult male presented with fatigue, brain fog, anxiety, and frequent bowel movements exceeding 10 movements per day, which were often watery and accompanied by urgency. The symptoms were consistent with CFS, anxiety, and irritable bowel syndrome. The patient reported having to nap on a daily basis, despite engaging in a seated job and extensive use of caffeinated beverages. Viral laboratories supported the presence of herpes 4 and 6 as persistent infections. Antiviral therapy with valacyclovir at 1000 mg two times per day orally was begun. The patient showed modest improvement in fatigue and the patient no longer needed to nap on a daily basis. However, patient still struggled with brain fog, anxiety, and frequent bowel movements. NTZ was added at 500 mg two times per day orally to the regimen. Within two months, the patient noted a marked improvement in fatigue, brain fog, and anxiety. In addition, the patient noted a marked reduction in the number of bowel movements he had each day from approximately 10 per day to 1-3 per day. After a year on the combination protocol, the patient was energetic, anxiety-free, and had normal bowel function.

### Example 2: Case Study 2

An adult woman presented with multiple symptoms of VIFD across multiple systems. She experienced fatigue, brain fog, and unrefreshing sleep consistent with CFS. She experienced muscle and joint pain with trigger point pain and exaggerated reactions to pain consistent with fibromyalgia. She experienced pelvic and vaginal pain, headaches, depression, anxiety, sore throat, painful lymph nodes, numbness, and generalized pain. The symptoms involved the central nervous system, the genitourinary system, the gastrointestinal system, the immune system, the musculoskeletal system and the peripheral nervous system. Laboratory testing revealed she had evidence of persistent Herpes 4 and 6 infections. The patient was started on valacyclovir at 1000 mg two times per day orally and over the course of three months minor improvement was seen in her symptoms. The dose of valacyclovir was increased to 2000 mg two times per day orally. Only slight additional benefit was realized. NTZ was then added at 500 mg two times per day orally. The patient showed slow but steady improvement in her symptoms over the subsequent 12 months.

### Example 3: Case Study 3

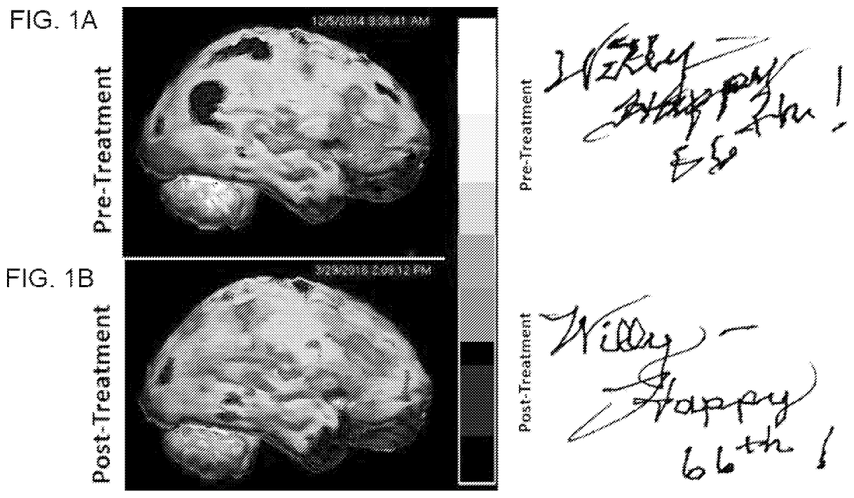

A late-middle-aged woman presented for evaluation of Parkinson's disease and depression. She had been diagnosed with Parkinson's disease by a neurologist based on clinical signs of tremor and bradykinesia. No neuroimaging had been done. After the evaluation, it was clear that neuroimaging would provide additional information. A single-photon emission computed tomography (SPECT) brain perfusion scan and a dopamine transporter SPECT scan (DaTscan) were ordered. The DaTscan revealed normal distribution of dopamine transporter and, thus, mitigating against the diagnosis of Parkinson's disease. The perfusion SPECT scan revealed diffuse hypoperfusion consistent with infectious or toxic injury to the brain (FIG. 1A). Laboratory testing revealed evidence for persistent Herpes 4 and 6 infections. The patient was started on antiviral therapy with valacyclovir at 1000 mg two times per day orally. The dose was increased to 1000 mg orally in the morning and 2000 mg orally at night. After three months, the patient noted a marked improvement in her depression symptoms. She also noted improved energy and motivation. However, she continued to experience tremor and bradykinesia. Therefore, NTZ was added at 500 mg two times per day orally. Over the next six months, the patient showed steady reduction in her tremor and improved mobility. After eight months on combination therapy, she was free of symptoms of Parkinson's disease and depression. A repeat perfusion SPECT brain scan was performed (FIG. 1B). The scan revealed marked improvement in brain function and near-complete resolution of the pathological findings associated with infectious injury.

### Example 4: Case Study 4

A 67 year old male presented for evaluation of memory problems. Cognitive testing was consistent with dementia. A SPECT functional brain scan was performed. The results were consistent with Alzheimer's disease. Laboratory testing revealed evidence of persistent Herpes 1 and Herpes 4 infections. The patient was started on antiviral therapy with valacyclovir 1000 mg two times per day orally. The valacyclovir was stopped and famciclovir was started at 500 mg two times per day orally. After one month, the famciclovir was increased to 1000 mg two times per day orally. After six months, the patient has shown modest improvement in memory function as reflected by improved scores on cognitive testing. However, he struggled still with daily memory function. NTX was added to his regimen at a dose of 500 mg two times per day orally. Within three months on a combination of valacyclovir and NTX, his memory function improved markedly. Family members were thrilled with the improvement.

### Example 5: Case Study 5

A 68 year old active woman had struggled with depression and obsessive-compulsive disorder in the past, but these problems were well-controlled with medications. She actively engaged in daily exercising, including skiing, bicycle riding, and vigorous walking. Suddenly, she developed severe fatigue. A medical workup did not reveal a cause. Laboratory testing revealed evidence of persistent Herpes 4 and Herpes 6 infections. The patient was started on valacyclovir at a dose of 1000 mg two times per day orally. After four months, the dose of valacyclovir was increased to 2000 mg two times per day, but her symptoms of severe fatigue did not improve. Then she was switched to famciclovir, titrating from 500 mg two times per day to 1000 mg two times per day orally. After three months with no improvement, she was switched to valganciclovir titrating from 450 mg two times per day to 900 mg two times per day. After four months without improvement, she was restarted on valacyclovir 1000 mg two times per day and NTX was added to her regimen at a dose of 500 mg two times per day orally. Within two months, the patient noted less fatigue. By six months, the patient was able to resume daily walking. At eight months of treatment, the patient was able to resume skiing.

### Example 6: Case Study 6

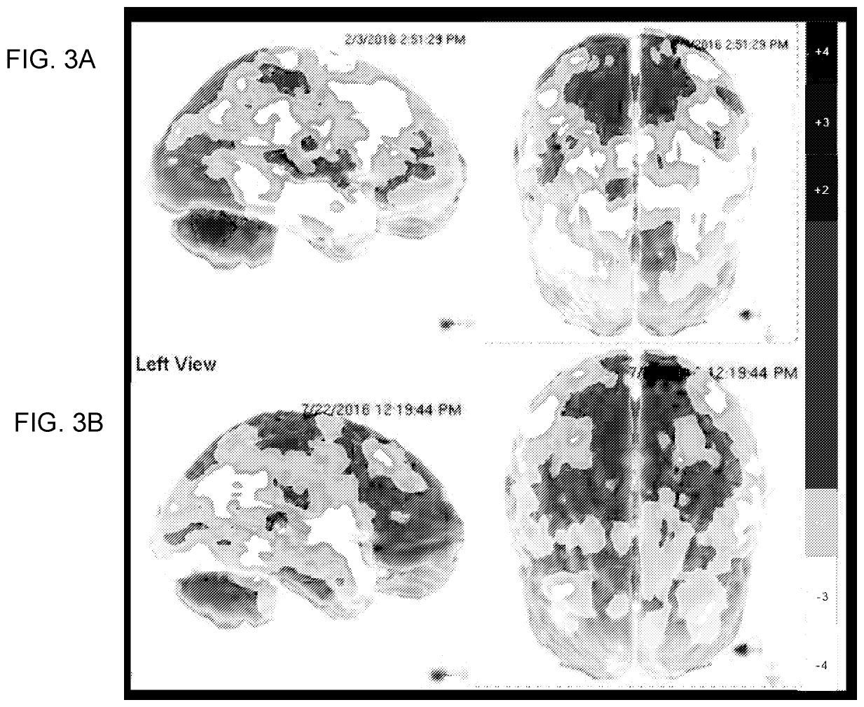

A 36 year old male presented with a history of depression since his teen years. He was severely depressed upon presentation (Quick Inventory of Depressive Symptomatology scale 26/27) and spent most of his time in his bedroom in the basement of his mother's house. He had withdrawn from college and had never held a full-time job. His condition was complicated by irritable bowel syndrome, brain fog, pervasive fatigue, and headaches. He had tried and failed to respond to more than 15 antidepressant medications. He then tried and failed to respond to ketamine infusion therapy. After failing to respond to the potent antidepressant, ketamine, he underwent laboratory testing which revealed high titers of antibodies to Epstein-Barr virus and to Herpes 1 virus. The patient also underwent a functional brain scan (single photon emission computed tomography—SPECT). The SPECT scan revealed pervasive underactivity of the cerebral cortices (FIG. 3). The patient was started on the antiviral valacyclovir at 1000 mg two times per day orally. After three months of treatment, he showed a roughly 20% improvement in symptoms. He was somewhat less fatigued and could think more clearly. He was somewhat less depressed. NTZ was added at a dose of 500 mg two times per day orally. After one month, the dose of NTZ was increased to 1000 mg two times per day orally. Within three months of the dose increase, the patient showed marked improvement in mood (Quick Inventory of Depressive Symptomatology scale 5/27). The IBS symptoms markedly improved. He no longer suffered with headaches and his energy and motivation greatly improved. A repeat SPECT scan showed marked improvement in cortical function (FIG. 3).

Claimed is:

1. A method of treating a virally induced functional disorder (VIFD) in a subject in need thereof, the method comprising administering to the subject a therapeutically effective amount of each of an antiviral compound and nitazoxanide.

2. A method of treating a functional somatic syndrome (FSS) in a subject in need thereof, the method comprising administering to the subject a therapeutically effective amount of each of an antiviral compound and nitazoxanide.

3. A method of treating a Herpes-family virus in a subject in need thereof, the method comprising administering to the subject a therapeutically effective amount of each of an antiviral compound and nitazoxanide.

4. The method of any one of claims 1-3, wherein the antiviral compound is an antiviral guanine analog.

5. The method of any one of claims 1-4, wherein the antiviral compound is valacyclovir, acyclovir, famciclovir, gangciclovir, or valganciclovir.

6. The method of any one of claims 1-5, wherein the antiviral compound is present in a unit dosage form from about 250 mg to about 6000 mg.

7. The method of claim 6, wherein the antiviral compound is present in a unit dosage form of 1000 mg.

8. The method of claim 6, wherein the antiviral compound is present in a unit dosage form of 2000 mg.

9. The method of any one of claims 1-8, wherein NTZ is present in a unit dosage form from about 10 mg to about 2000 mg.

10. The method of claim 9, wherein NTZ is present in a unit dosage form from of 500 mg.

11. The method of claim 9, wherein NTZ is present in a unit dosage form from of 250 mg.

12. The method of claim 9, wherein NTZ is present in a unit dosage form from of 1000 mg.

13. The method of any one of claims 1-4, wherein the antiviral compound and NTZ is administered in a dose weight ratio range from about one-to-one to about five hundred-to-one of the antiviral compound to NTZ.

14. The method of any one of claims 1-13, wherein the antiviral compound is administered to the subject twice a day.

15. The method of any one of claims 1-14, wherein NTZ is administered to the subject twice a day.

16. The method of claim 1, wherein the virally induced functional disorder (VIFD) is a disorder of the genitourinary system, a disorder of the gastrointestinal system, a disorder of the central nervous system, a disorder of the immune system, a disorder of the musculoskeletal system, or a disorder of the peripheral nervous system.

17. The method of claim 2, wherein the functional somatic syndrome (FSS) is irritable bowel syndrome (IBS), chronic fatigue syndrome (CFS), fibromyalgia, multiple chemical sensitivity, muscle pain, joint pain, nonspecific chest pain, premenstrual syndrome, non-ulcer dyspepsia, repetitive strain injury, headache, temporomandibular joint disorder, chronic pain, chronic pelvic pain, atypical facial pain, low back pain, sick building syndrome, Gulf War syndrome, interstitial cystitis, chronic Lyme disease, depression, post-traumatic stress disorder (PTSD), chronic anxiety disorder, brain fog, cognitive dysfunction, unrefreshing sleep, insomnia, dizziness and/or tinnitus.

18. A pharmaceutical composition comprising a therapeutically effective amount of each of an antiviral compound and nitazoxanide.

19. The pharmaceutical composition of claim 18, wherein the antiviral compound is an antiviral guanine analog.

20. The pharmaceutical composition of any one of claims 18-19, wherein the antiviral compound is valacyclovir, acyclovir, famciclovir, gangciclovir, or valganciclovir.

21. The pharmaceutical composition of any one of claims 18-20, wherein the antiviral compound is present in a unit dosage form from about 250 mg to about 6000 mg.

22. The pharmaceutical composition of claim 21, wherein the antiviral compound is present in a unit dosage form of 1000 mg.

23. The pharmaceutical composition of claim 21, wherein the antiviral compound is present in a unit dosage form of 2000 mg.

24. The pharmaceutical composition of any one of claims 18-23, wherein NTZ is present in a unit dosage form from about 10 mg to about 2000 mg.

25. The pharmaceutical composition of claim 24, wherein NTZ is present in a unit dosage form of 500 mg.

26. The method of claim 25, wherein NTZ is present in a unit dosage form from of 250 mg.

27. The method of claim 25, wherein NTZ is present in a unit dosage form from of 1000 mg.

28. The pharmaceutical composition of claim 18, wherein the antiviral compound to nitazoxanide is present in a dose weight ratio range from about one-to-one to about five hundred-to-one.
